(19) United States
(12) Patent Application Publication
(10) Pub. No.: US 2013/0304228 A1
(43) Pub. Date: Nov. 14, 2013

(54) BONE JOINT PROSTHESIS AND METHOD

(71) Applicant: ZIMMER, INC., Warsaw, IN (US)

(72) Inventor: James P. Phipps, South Whitley, IN (US)

(73) Assignee: Zimmer, Inc., Warsaw, IN (US)

(21) Appl. No.: 13/887,686

(22) Filed: May 6, 2013

Related U.S. Application Data
(60) Provisional application No. 61/644,229, filed on May 8, 2012.

(51) Int. Cl. A61F 2/36

(52) U.S. Cl. CPC A61F 2/3601
USPC 623/23.42

(57) ABSTRACT

A bone joint prosthesis can augment or replace a natural bone joint. The prosthesis can include a joint head portion and an elongated stem. The stem can include a proximal end coupled to the joint head portion and a distal end having a sharp edge. A stem body, including a bore, can extend between the proximal end of the elongated stem and the sharp edge at the distal end of the elongated stem. The sharp edge of the elongated stem can be capable of being pressed into a long bone so as to core intact marrow into the bore of the stem body. A method can include preparing a bone for a bone joint prosthesis. A sharp distal end of an elongated stem of the bone joint prosthesis can be pressed into the bone. Intact bone marrow can be cored into a bore of the elongated stem.

### CLAIM OF PRIORITY

This application claims the benefit of priority under 35 U.S.C. §119(e) of Phipps, U.S. Provisional Patent Application Ser. No. 61/644,229, entitled “BONE JOINT PROSTHESIS AND METHOD”, filed on May 8, 2012, which is herein incorporated by reference in its entirety.

### BACKGROUND

A bone joint prosthesis can augment or replace one or more portions of a joint. Particularly, in a “total” arthroplasty procedure, the entire joint can be replaced with prosthetic components. Conversely, in a “partial” arthroplasty procedure, only a portion of the joint can be replaced with a prosthetic component. Partial arthroplasty procedures can rely on existing bone surfaces to mate and articulate with the prosthetic component. Arthroplasty procedures, whether total or partial, can function to fix damaged joints as well as relieve the pain associated with arthritis.

For example, a knee joint prosthesis can replace one or more portions of a distal femur and/or a proximal tibia. A shoulder joint prosthesis can replace one or more portions of a proximal humerus and/or a glenoid fossa of a scapula. A hip joint prosthesis can replace one or more portions of a proximal femur and/or an acetabulum of a pelvis. An elbow joint prosthesis can replace one or more portions of the ulna and/or humerus.

U.S. Pat. No. 4,595,393 is directed toward a hip joint prosthesis having a hollow stem.

U.S. Pat. No. 4,784,124 is directed toward a prosthetic bone implant and a tool for inserting the implant into a bone.

U.S. Pat. No. 5,330,536 is directed toward a femur portion of a hip prosthesis.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view illustrating an example of a bone joint prosthesis;

FIG. 2 is a reverse configuration perspective view the bone joint prosthesis of FIG. 1; and

FIG. 3 is a flow chart illustrating an example of a method of inserting a bone joint prosthesis into a cavity.

### DETAILED DESCRIPTION

The present disclosure relates generally to a bone joint prosthesis and related method of use. The bone joint prosthesis can include an elongated stem having a distal end with a sharp edge configured so as to help ease the elongated stem into a bone. The sharp edge can, for example, be configured to provide a benefit of directing intact marrow within the bone into a bore of the elongated stem. The elongated stem can include one or more orifices configured so as to help support ingrowth of the bone and the marrow within the bore. Benefits of such examples can include, but are not limited to, reducing disruption to existing marrow within the bone, preserving current bone within a subject, and improving prosthesis fixation.

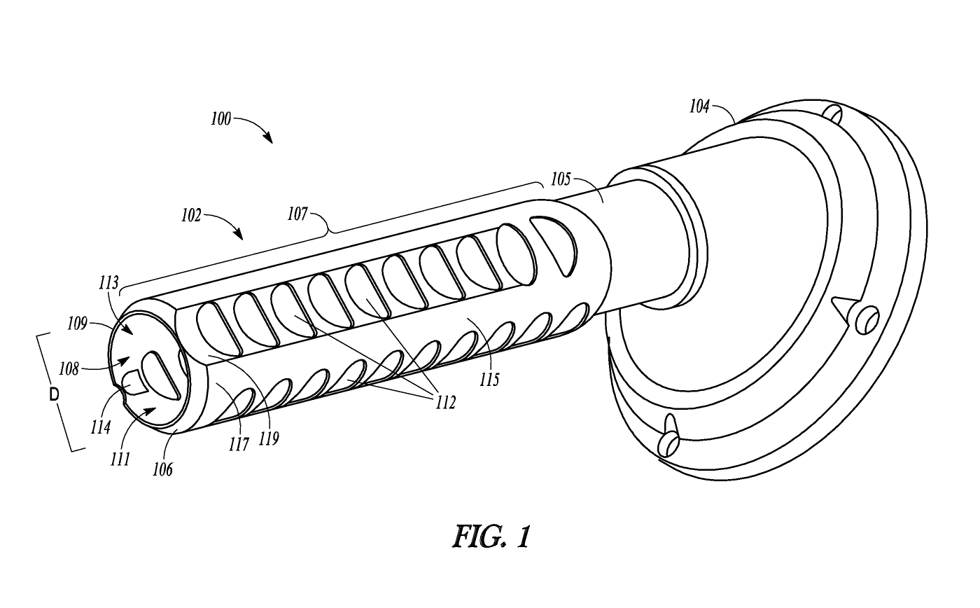

FIG. 1 is a perspective view illustrating an example of a bone joint prosthesis 100. The bone joint prosthesis 100 can include an elongated stem 102 and a joint head portion 104. The elongated stem 102 of the bone joint prosthesis 100 can be sized, shaped, or otherwise configured as a stem for receipt within a bone. The bone can include a natural, anatomical cavity, such as an intramedullary canal. In various examples, the elongated stem 102 can be configured as a humeral stem for use in a shoulder arthroplasty procedure, a femoral stem for use in a hip or knee arthroplasty procedure, a tibial stem for use in a knee arthroplasty procedure, or a humeral stem or ulnar stem for use in an elbow arthroplasty procedure. The joint head portion 104 can be sized, shaped, or otherwise configured for replacement of various bone portions, such as a humeral socket for use in a shoulder arthroplasty procedure, a femoral head for use in a hip arthroplasty procedure, a femoral component for use in a knee arthroplasty procedure, or a tibial plateau for use in a knee arthroplasty procedure.

The elongated stem 102 and the joint head portion 104 can comprise any suitable biocompatible material. Examples include, but are not limited to, titanium or a cobalt chromium alloy. The elongated stem 102 can include a proximal end 105 coupled to the joint head portion 104, a distal end 106, and a stem body 107 extending between the proximal end 105 and the distal end 106. The proximal end 105 of the elongated stem 102 can be integral with or detachably coupled to the joint head portion 104. Any suitable connection means can be used including, for example, welding, a snap-fit connection, a compression-fit connection, one or more bolts, an adhesive, or the like. The proximal end 105 can include a tapered portion connecting the stem body 107 to the joint head portion 104. The tapered portion can be solid or include a hollow portion.

The distal end 106 of the elongated stem 102 can include a sharp edge 109, configured so as to separate a material from or within a bone. The sharp edge 109 can be sharp enough to cut marrow or bone. For example, the sharp edge 109 can be configured to cleave marrow from a marrow-bone interface or core intact marrow from a marrow cavity. The sharp edge 109 can be configured as, for example, a serrated edge, a rounded edge, a rounded fillet-type edge, or combinations thereof. Providing the sharp edge 109 on the elongated stem 102 can help to ease the insertion of the stem body 107 into a bone, such as a marrow cavity.

The stem body 107 can be generally cylindrical in shape and have a substantially circular cross-section. However, other cross-section shapes are also contemplated, such as square, rectangular, triangular, ovular, or the like. The stem body 107 can be configured as a hollow tube having a bore 108 extending at least a portion between the proximal end 105 of the elongated stem 102 and the distal end 106 of the elongated stem 102. The bore 108 can provide a means for preserving existing marrow within the bone. Benefits of such an example can include reducing disruption of existing bone marrow within the bone, increasing fixation of the prosthesis 100 within the subject, and increasing quality of life of a subject by helping to provide a more natural joint replacement. The bore 108 can have a cross-sectional shape matching that of the stem body 107, or a cross-sectional shape that is different than the stem body 107. The bore 108 can extend along the entire axial length between the proximal end 105 and the distal end 106 of the elongated stem 102, or can extend along only a portion of the axial length. For example, FIG. 1 shows a bore 108 that extends from the proximal end 105 of the elongated stem 102 all the way through an opening 111 formed in the distal end 106. The axial length of the bore 108 can be based on the desired amount of bone marrow to be captivated in the bore or the desired amount of bone in-growth into the elongated stem 102.

The stem body 107 can include one or more orifices 112 extending from an outer surface of the stem body 107 into the bore 108. The one or more orifices 112 can be sized and shaped to permit bone or marrow growth between a region outside of the stem body 107 and the bore 108 of the stem body 107. The one or more orifices 112 do not have to be, but can be, uniform; that is, they can vary in size or shape. An inner surface 113 of the stem body 107, which defines the outer boundary of the bore 108, can include one or more grooves 114 extending along at least a portion of the axial length of the stem body 107. One or more of the orifices 112 can be provided along the groove 114. Benefits of such an example can include increasing a total volume of the bore, so as to be capable of accepting a greater volume of marrow within the bore 108. The groove 114 can be configured to permit a greater amount of bone marrow into the bore 108. The grove 114 can provide the benefit of increasing the volume within the bore or providing a greater surface area of the bore for increased ingrowth. Generally speaking, the greater the internal stem volume defined by the bore 108, the greater the amount of bone marrow that can be permitted into the stem body 107. This can increase bone in-growth between the elongated stem 102 and the long bone by providing a greater surface area for a marrow or bone and elongated stem material interface.

In an example, the elongated stem 102 can be configured to be inserted into a marrow cavity of a long bone and to reside within the marrow cavity after insertion. The elongated stem 102 can have a lateral internal dimension, such as inner diameter D, that can be smaller than a lateral dimension of the bone marrow cavity of the long bone, so as to allow for coring of a substantially intact sample of marrow from within the bone into the bore 108 of the stem body 107. In an example, the stem body 107 can be provided with an elongated stem 102.

The plurality of orifices 112 can be distributed along at least a portion of the axial length of the stem body 107. FIG. 1 shows a plurality of orifices 112 distributed substantially along the entire length of the stem body 107. Distribution of the orifices 112 along the stem body 107 can be uniform or non-uniform. In an example, an anterior side of the hollow tube can include a plurality of orifices 112 along a portion of the axial length of the stem body 107 and a lateral side can include a plurality of orifices 112 along substantially the entire length of the stem body 107. In an example, the plurality of orifices 112 can be configured in an offset or random pattern that circumscribes at least a portion of the hollow tube.

The one or more orifices 112 can be configured to distribute an impact force during insertion of the elongated stem 102 into a cavity while reducing the risk of fracturing the bone. The sharp edge 109 at the distal end 106 of the elongated stem 102 can be configured to help guide the stem body 107 as it is pressed into the cavity, and can assist with coring the marrow into the bore 108 of the stem body 107. The sharp edge 109 can also function to deter bone marrow from separating into distinct pieces or flowing outside of the bore 108 of the stem body 107 during insertion of the elongated stem 102 into the cavity.

The inner lateral dimension D of the bore at the sharp-edged distal end 106 can be greater than an outer lateral dimension of an abutting end of a device pre-inserted into the long bone. For example, the inner lateral dimension D of the sharp-edged distal end 106 of the elongated stem 102 of a shoulder joint prosthesis can be greater than an outer dimension (e.g., outer diameter) of an abutting end of a pre-inserted elbow joint, so as to permit the abutting end to fit within the sharp-edged distal end 106.

The outer surface 115 of the stem body 107 can define a smooth, uniform wall surface or a contoured wall surface. In an example, as illustrated in FIG. 1, the outer surface of the stem body 107 can include a portion having a convex wall surface 117 and a portion having a concave tube wall surface 119. Providing such convex 117 and concave 119 wall surfaces can assist with pressing the elongated stem 102 into the cavity by helping to distribute a force used to press the distal end into the bone. The concave wall surfaces 119 can increase surface area of the outer surface of the stem body 107, which can increase the potential for ingrowth between the stem body 107 and the marrow or bone.

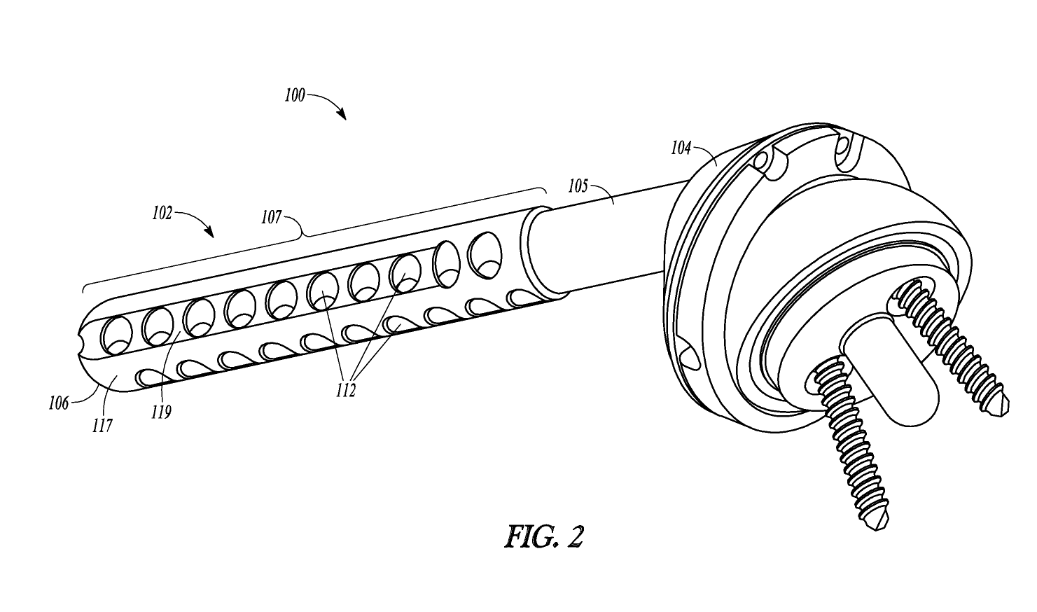

FIG. 2 is a reverse configuration perspective view the bone joint prosthesis 100. The joint head portion 104 can connect to glenoid sphere and a glenoid fixation device with two screws, such as with a reverse should joint prosthesis. In such an example, the elongated shaft 102, as described herein, can include a humeral stem; including, for example, convex 117 or concave 119 surfaces on at least a portion of the stem body 107 between the sharp-edged distal 106 and proximal 105 ends.

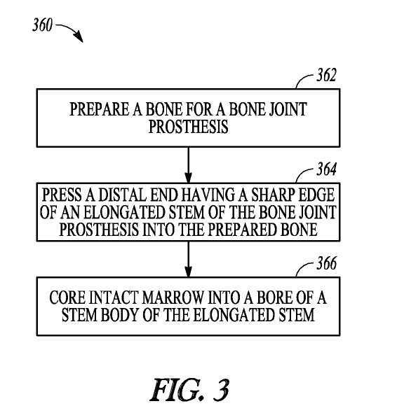

FIG. 3 is a flow chart illustrating an example of a method 360 for inserting a bone joint prosthesis into a bone cavity. At 362, a long bone can be prepared for a bone joint prosthesis. Preparation can include, for example, making one or more bone cuts, forming a cavity in a bone, clearing debris from a bone cavity, or the like.

At 364, a bone prosthesis having an elongated stem with a sharp-edged distal end can be pressed into the prepared bone. The sharp-edged distal end can be pressed by use of a hammer, an impaction drill, or combinations thereof. In an example, the sharp-edged distal end can be pressed so as to contact cortical bone.

At 366, intact marrow can be cored into a bore of the elongated stem, so as to remove a substantially intact portion of marrow from the bone. In an example, the marrow from the bone can be cleaved into the bore of the elongated stem, so as to separate marrow at an interface of the marrow and the bone.

In an example, the bone or marrow can be permitted to grow through a plurality of orifices connecting a region outside of the elongated stem and a region within the bore of the elongated stem.

Each of these non-limiting examples can stand on its own, or can be combined in any permutation or combination with any one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A bone joint prosthesis, comprising:
a joint head portion; and
an elongated stem, the elongated stem including:
a proximal end configured to be coupled to the joint head portion;
a distal end having a sharp edge; and
a stem body, including a bore, the bore extending between the proximal end of the elongated stem and the sharp edge at the distal end of the elongated stem, wherein the sharp edge is capable of being pressed into a bone so as to core intact marrow into the bore of the stem body.

2. The prosthesis of claim 1, wherein the stem body includes a plurality of orifices that are sized and shaped to permit bone or marrow growth between a region outside of the stem body and a region within the bore of the stem body.

3. The prosthesis of claim 2, wherein where the plurality of orifices are distributed along at least half of a length of the stem body.

4. The prosthesis of claim 2, wherein the plurality of orifices are configured in an offset pattern that circumscribes at least a portion of the stem body.

5. The prosthesis of claim 2, wherein the plurality of orifices are configured to distribute an impact force such that the elongated stem is capable of being pressed into the bone.

6. The prosthesis of claim 1, wherein the sharp edge at the distal end of the elongated stem is a chamfered edge.

7. The prosthesis of claim 1, wherein the sharp edge at the distal end of the elongated stem is a serrated edge.

8. The prosthesis of claim 1, wherein the sharp edge at the distal end of the elongated stem is configured to cleave the marrow from the bone into the bore of the stem body.

9. The prosthesis of claim 1, wherein the stem body is configured to reside within a marrow region of the bone.

10. The prosthesis of claim 1, wherein an inner lateral dimension of the bore at the distal end of the elongated stem is greater than an outer lateral dimension of an abutting end of a device pre-inserted into the bone.

11. The prosthesis of claim 1, wherein the stem body includes a groove extending along at least a portion of a length of the stem body.

12. The prosthesis of claim 11, wherein one or more of the plurality of orifices is positioned along the groove.

13. The prosthesis of claim 1, wherein the bore extends at least half of a length of the stem body.

14. The prosthesis of claim 1, wherein the elongated stem of the bone joint prosthesis is sized, shaped, or otherwise configured as a femoral stem or a humeral stem.

15. The prosthesis of claim 1, wherein a wall of the elongated stem includes a convex tube wall portion and a concave tube wall portion.

16. A method, comprising:
preparing a bone for a bone joint prosthesis;
pressing a sharp distal end of an elongated stem of the bone joint prosthesis into the prepared bone; and
coring intact marrow into a bore of a stem body of the elongated stem.

17. The method of claim 16, including permitting bone or marrow growth through a plurality orifices connecting a region outside of the stem body and a region within the bore of the stem body.

18. The method of claim 16, wherein pressing the sharp distal end includes contacting cortical bone.

19. The method of claim 16, including cleaving the marrow from the bone into the bore of the stem body.

20. A bone joint prosthesis, comprising:
a joint head portion; and
an elongated stem, the elongated stem including:
a proximal end configured to be coupled to the joint head portion;
a distal end having a sharp edge; and
a stem body, including a bore, the bore extending between the proximal end of the elongated stem and the sharp edge at the distal end of the elongated stem:
wherein the distal end is configured to be pressed into a bone so as to core intact marrow into the bore of the stem body; and
wherein the stem body includes a tube wall having a plurality orifices sized and shaped to permit bone or marrow growth between a region outside of the stem body and a region within the bore of the stem body.
